(19) United States
(12) Patent Application Publication
(10) Pub. No.: US 2019/0298291 A1
(43) Pub. Date: Oct. 3, 2019

(54) BRAIN IMAGING SYSTEM AND METHOD

(71) Applicant: A-moy Limited, Apia (WS)

(72) Inventor: FAN-PEI YANG, Hsinchu City (TW)

(21) Appl. No.: 16/366,431

(22) Filed: Mar. 27, 2019

(30) Foreign Application Priority Data
Mar. 30, 2018 (TW) 107111316

(51) Int. Cl. A61B 6/00; A61B 6/03; A61B 5/055; G06K 9/62

(52) U.S. Cl. CPC A61B 6/501; A61B 6/481; A61B 6/032; G06K 9/6296; A61B 6/504; A61B 6/5247; A61B 5/055; A61B 6/507

(57) ABSTRACT

A brain imaging system includes a first imaging device, a second imaging device, a third imaging device and a central processor. The first imaging device captures a first brain image, and calculates a cerebral blood flow, a cerebral blood volume, a cerebral blood mean transit time and a first contrast agent time to peak. The second imaging device captures a second brain image, and calculates the cerebral blood flow, the cerebral blood volume, the cerebral blood mean transit time and a second contrast agent time to peak. The central processor generates an image of a vessel occlusion, infarction or ischemia region respectively according to the vessel occlusion, infarction or ischemia regions in the first brain image and in the second brain image. The third imaging device obtains a brain atrophy region according to the cerebral cortex volume calculated by the third imaging device.

### CROSS-REFERENCE TO RELATED PATENT APPLICATION

This application claims the benefit of priority to Taiwan Patent Application No. 107111316, filed on Mar. 30, 2018, now Taiwan Patent No. 1644653. The entire content of the above identified application is incorporated herein by reference.

### BACKGROUND OF THE INVENTION

### 1. Field of the Invention

The present disclosure relates to an imaging system and an imaging method; in particular, to a brain imaging system and a brain imaging method.

### 2. Description of Related Art

Nuclear magnetic resonance (NMR) is a non-invasive way to detect human bodies. It obtains variations of magnetic dipole moment of water molecules through transmitting and receiving radio frequency signals, and further to differentiate normal and tumor tissues by using contrast agents. The computerized tomography (CT) is used to obtain a two-dimensional image by having X-rays scanned through a human body. However, the two-dimensional image can only be interpreted by medical staff, which may adversely affect the precision and efficiency thereof.

### SUMMARY OF THE INVENTION

The present disclosure provides a brain imaging system for capturing a brain image by using contrast agents. The brain imaging system detects positions of an entrance and an exit where the contrast agents flow into and out from a brain, and includes a first imaging device, a second imaging device, a third imaging device and a central processor. The first imaging device is configured to capture a first brain image, convert the first brain image to a first concentration curve by Hounsfield Unit, and calculate a cerebral blood flow, a cerebral blood volume, a cerebral blood mean transit time and a first contrast agent time to peak according to the first concentration curve showing concentrations at the positions of the entrance and the exit. The second imaging device is configured to capture a second brain image, convert the second brain image to a second concentration curve through an equation, and calculate the cerebral blood flow, the cerebral blood volume, the cerebral blood mean transit time and a second contrast agent time to peak according to the second concentration curve showing concentrations at the positions of the entrance and the exit. The third imaging device is configured to calculate a cerebral cortex volume. The central processor is coupled to the first imaging device, the second imaging device and the third imaging device, and is configured to detect a vessel occlusion, infarction or ischemia region of the first brain image according to at least one of the cerebral blood flow, the cerebral blood volume, the cerebral blood mean transit time and the first contrast agent time to peak, and detect a vessel occlusion, infarction or ischemia region of the second brain image at least according to the second concentration curve, the cerebral blood flow, the cerebral blood volume, the cerebral blood mean transit time or the second contrast agent time to peak. The central processor generates an image of the vessel occlusion, infarction or ischemia region respectively according to the vessel occlusion, infarction or ischemia region in the first brain image and the vessel occlusion, infarction or ischemia region in the second brain image. The third imaging device obtains a brain atrophy region according to the cerebral cortex volume.

The present disclosure also provides a brain imaging method adapted to a brain imaging system for capturing a brain image by using contrast agents. The brain imaging system detects positions of an entrance and an exit where the contrast agent flows into and out from a brain. The brain imaging system includes a first imaging device, a second imaging device, a third imaging device and a central processor, and the central processor is coupled to the first imaging device, the second imaging device and the third imaging device. The brain imaging method includes: through the first imaging device, capturing a first brain image; through the first imaging device, converting the first brain image to a first concentration curve by the Hounsfield unit; through the first imaging device, calculating a cerebral blood flow, a cerebral blood volume, a cerebral blood mean transit time and a first contrast agent time to peak according to the first concentration curve showing concentrations at the positions of the entrance and the exit; through the second imaging device, capturing a second brain image; through the second imaging device, converting the second brain image to a second concentration curve through an equation; through the second imaging device, calculating the cerebral blood flow, the cerebral blood volume, the cerebral blood mean transit time and a second contrast agent time to peak; through the third imaging device, calculating a cerebral cortex volume; through the central processor, detecting a vessel occlusion, infarction or ischemia region of the first brain image according to one of the cerebral blood flow, the cerebral blood volume, the cerebral blood mean transit time or the first contrast agent time to peak; and through the central processor, detecting a vessel occlusion, infarction or ischemia region of the second brain image according to one of the second concentration curve, the cerebral blood flow, the cerebral blood volume, the cerebral blood mean transit time and the second contrast agent time to peak. The central processor generates an image of the vessel occlusion, infarction or ischemia region respectively according to the vessel occlusion, infarction or ischemia region in the first brain image and the vessel occlusion, infarction or ischemia region in the second brain image, and the third imaging device obtains a brain atrophy region according to the cerebral cortex volume.

For further understanding of the present disclosure, reference is made to the following detailed description illustrating the embodiments of the present disclosure. The description is only for illustrating the present disclosure, not for limiting the scope of the claim.

### BRIEF DESCRIPTION OF THE DRAWINGS

Embodiments are illustrated by way of example and not by way of limitation in the figures of the accompanying drawings, in which like references indicate similar elements and in which:

FIG. 1A shows a block diagram of a brain imaging system according to one embodiment of the present disclosure;

FIG. 1B is a schematic diagram showing a flow path of a contrast agent according to one embodiment of the present disclosure;

FIG. 1C is a curve diagram showing an accumulated concentration function of a contrast agent according to one embodiment of the present disclosure;

FIG. 1D is a curve diagram showing a residual concentration function of a contrast agent according to one embodiment of the present disclosure;

FIG. 1E shows a schematic diagram of a brain image according to one embodiment of the present disclosure; and

FIG. 2 shows a flow chart of a brain imaging method according to one embodiment of the present disclosure.

### DETAILED DESCRIPTION OF EXEMPLARY EMBODIMENTS

The aforementioned illustrations and following detailed descriptions are exemplary for the purpose of further explaining the scope of the present disclosure. Other objectives and advantages related to the present disclosure will be illustrated in the subsequent descriptions and appended drawings. In these drawings, like references indicate similar elements.

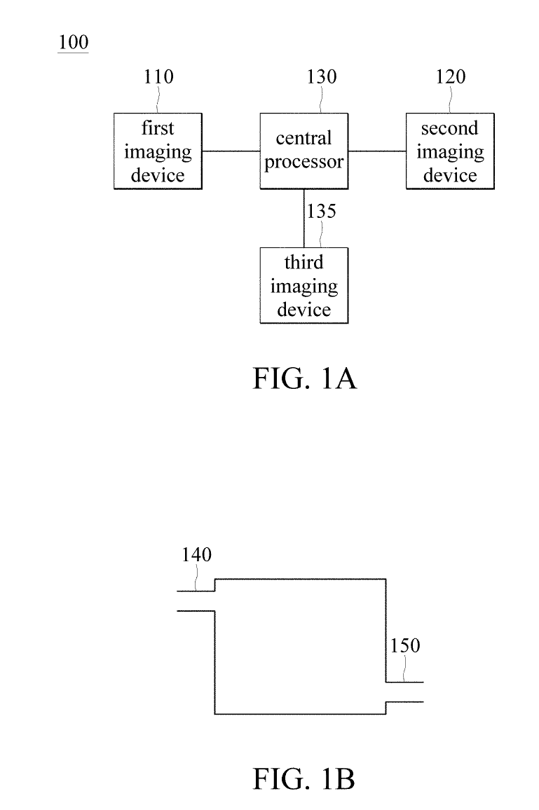

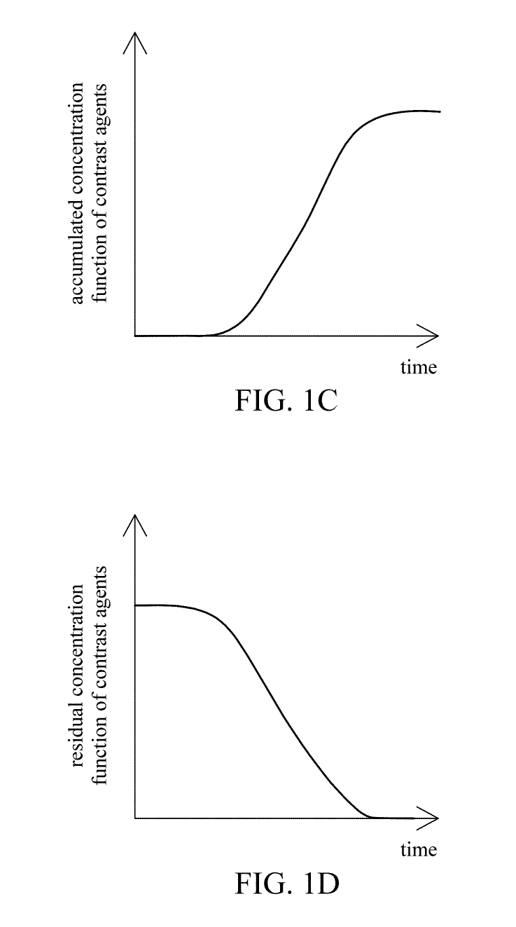

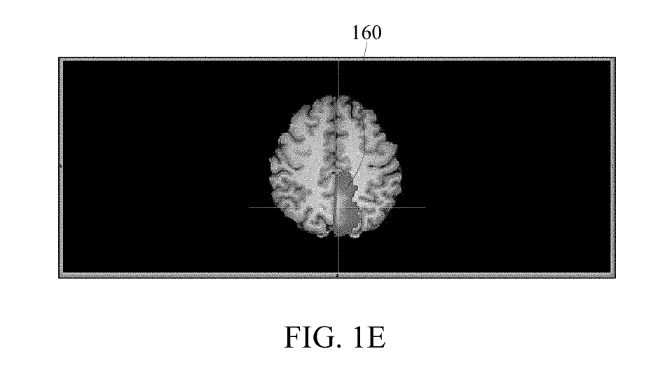

FIG. 1A shows a block diagram of a brain imaging system according to one embodiment of the present disclosure, FIG. 1B is a schematic diagram showing a flow path of contrast agents according to one embodiment of the present disclosure, FIG. 1C is a curve diagram showing an accumulated concentration function of a contrast agent according to one embodiment of the present disclosure, FIG. 1D is a curve diagram showing a residual concentration function of a contrast agent according to one embodiment of the present disclosure, and FIG. 1E shows a schematic diagram of a brain image according to one embodiment of the present disclosure.

A brain imaging system 100 includes a first imaging device 110, a second imaging device 120, a third imaging device 135 and a central processor 130. The brain imaging system 100 captures a brain image by using a contrast agent. The brain imaging system 100 detects positions of an entrance 140 and an exit 150 where the contrast agent flows into and out from a brain. The first imaging device 110 captures a first brain image, and converts the first brain image to a first concentration curve by Hounsfield Unit (HU). The first imaging device 110 calculates a cerebral blood flow (CBF), a cerebral blood volume (CBV), a cerebral blood mean transit time (MTT) and a first contrast agent time to peak (TTP) according to the first concentration curve showing concentrations at the positions of the entrance 140 and the exit 150.

Specifically, the first imaging device 110 is a computed tomography (CT) imaging device, and the first brain image is a CT brain image. In this embodiment, the first concentration curve is a concentration curve of an iodinated contrast agent, and the first contrast agent time to peak is an iodinated contrast agent time to peak. A slope of the concentration curve of the iodinated contrast agent is positively proportional to Hounsfield Unit. The central processor 130 detects the position of the entrance 140 of the brain according to an iodinated contrast agents starting time, an iodinated contrast agents time to half-peak and the iodinated contrast agent time to peak.

The second imaging device 120 captures a second brain image, and converts the second brain image to a second concentration curve through an equation. The second imaging device 120 calculates the cerebral blood flow, the cerebral blood volume, the cerebral blood mean transit time and a second contrast agent time to peak according to the second concentration curve showing concentrations at the positions of the entrance 140 and the exit 150 of the brain. The third imaging device 135 captures a third brain image. The central processor 130 uses an FMRIB Software Library (FSL) software to capture the third brain image. The third brain image is a structural brain image, and the structural brain image is a T1 image. In addition, the third imaging device 135 can be also configured to calculate a cerebral cortex volume to obtain a brain atrophy region. For example, the cerebral cortex may be the parietal cortex, the frontal lobe, the temporal cortex or the occipital lobe.

Specifically, the second imaging device 120 is a magnetic resonance imaging (MRI) device, and the second brain image is an MRI brain image. In this embodiment, the second concentration curve is a concentration curve of a Gadolinium contrast agent, and the second contrast agent time to peak is a Gadolinium contrast agent time to peak. The central processor 130 detects the position of the entrance 140 of the brain according to a Gadolinium contrast agent starting time, a Gadolinium contrast agent time to half-peak and the Gadolinium contrast agent time to peak. It should be noted that, the Gadolinium contrast agent can be Gadolinium-DiethyleneTriamine Penta-acetic Acid (Gd-DTPA). Since Gd³⁺ in the lanthanide series is toxic and may lead to renal fibrosis as the excessive Gd³⁺ accumulates in human bodies, the Gd³⁺ is chelated by DTPA to form a stable compound, the Gd-DTPA. FIG. 1C and FIG. 1D show that, the accumulated concentration function of the contrast agent increases but the residual concentration function of the contrast agent decreases with time.

The central processor 130 detects a vessel occlusion, infarction or ischemia region of the first brain image according to one of the cerebral blood flow, the cerebral blood volume, the cerebral blood mean transit time and the first contrast agent time to peak. Specifically, when the cerebral blood flow is below 30% of a normal cerebral blood flow, the cerebral blood volume is smaller than 40% of a normal cerebral blood volume and the first contrast agent time to peak is increasing, the central processor 130, through the first imaging device 110, detects an infarct core of the vessel occlusion, infarction or ischemia region in the first brain image. In addition, when the cerebral blood flow is decreasing, the cerebral blood volume is maintained or increased, and the first contrast agent time to peak is dramatically increasing, the central processor 130 through the first imaging device 110 detects a penumbra of the vessel occlusion, infarction or ischemia region in the first brain image.

The central processor 130 detects a vessel occlusion, infarction or ischemia region of the second brain image through an equation and according to one of the second concentration curve, the cerebral blood flow, the cerebral blood volume, the cerebral blood mean transit time and the second contrast agent time to peak. Furthermore, the equation can be as below.

$$S ( x , y , b ) = {M}_{0} ( 1 - {e}^{- \frac{TR}{{T}_{1} ( x , y )}} ) {e}^{- \frac{TE}{{T}_{2}^{*} ( x , y )}} {e}^{- ADC \cdot b}$$

T₁ is a spin-lattice relaxation time, T₂* is a transverse relaxation time, TR is a cycle time, TE is an echo time, b is a setting parameter of an imaging device, (x, y) is a position of the brain image, and Mo is an initial value of the brain image when time is zero). In practice, “b” can be set as 0 or 1000. T₁ is parallel with a magnetic field orientation. When a magnetic dipole moment is opposite to the magnetic field orientation, the magnetic dipole moment has a maximum energy. On the other hand, when the magnetic dipole moment and the magnetic field orientation are the same, the magnetic dipole moment has a minimum energy. T₂* is vertical to the magnetic field orientation. Generally, a substance includes magnetic dipole moments, and each of them has different energy with respect to the magnetic field. Some magnetic dipole moments have higher energy, and some magnetic dipole moments have lower energy. The vector sum of all the magnetic dipole moments will gradually decrease, and a decreasing rate can be represented by the transverse relaxation time T₂*.

The equation can be considered a diffusion weighted image (DWI). A substance diffusion is 3-dimensional. A diffusion of water molecules may be affected by the surroundings and other molecules close to them, and thus the diffusion of water molecules is anisotropic. Fractional anisotropy (FA) is a value to evaluate the anisotropy of the molecule diffusion. The FA is a value from 0 to 1, wherein “1” indicates a high degree of anisotropy but “0” indicates a low degree of anisotropy. For example, a white matter has a high degree of anisotropy, but a grey matter has a low degree of anisotropy.

The above equation includes an apparent diffusion coefficient (ADC) as follows.

$$ADC ( x , y ) = - \frac{1}{b} ln ( \frac{S ( x , y , 1000 )}{S ( x , y , 0 )} ) {mm}^{2} \text{/} s$$

When the ADC is smaller than the diffusion threshold, the central processor 130 through the second imaging device 120 detects an infarct core of the vessel occlusion, infarction or ischemia region in the second brain image. In practice, the ADC should be divided by 1,000,000, and the position of the brain image (x, y) includes two algebras referring to the position of the brain image. For example, the diffusion threshold can be 600 mm²/s. When the second contrast agent time to peak is larger than a time to peak, the central processor 130 through the second imaging device 120 detects a penumbra of the vessel occlusion, infarction or ischemia region in the second brain image. The time to peak may be 6 seconds. The diffusion threshold and the time to peak are calculated by the central processor 130 based on Bayesian statistics, but values are not restricted herein.

In FIG. 1E, the central processor 130 uses an FMRIB Software Library (FSL) software to execute a Brain Extraction Tool to capture a calvarium image of the second brain image and separate the calvarium image from the second brain image. Then, the central processor 130 divides the second brain image without the calvarium image into a plurality of brain regions. The central processor 130 detects a penumbra 160 of the vessel occlusion, infarction or ischemia region in the second brain image based on the Bayesian statistics. Specifically, according to a FSL instruction, the central processor 130 divides the second brain image without the calvarium image into 15 brain regions, wherein these 15 brain regions include left brain regions and right brain regions. When the central processor 130 receives the FSL instruction, the central processor 130 uses the FSL software to do calculations for the cortex division, positions of the brain regions and volumes of the brain regions. The diffusion thresholds of the brain regions are different. Also, the diffusion thresholds of the brain regions may be varied due to age, gender or brain diseases. The central processor 130 can determines the diffusion thresholds of all brain regions based on a big data analysis (e.g., the Bayesian statistics) to detect the penumbra 160 of the vessel occlusion, infarction or ischemia region in the second brain image. Therefore, by using the FSL software, the central processor 130 can not only divide the calvarium image from the second brain image, but also can calculate the volume of each brain region.

The central processor 130 uses algorithms to generate an image of the vessel occlusion, infarction or ischemia region according to the vessel occlusion, infarction or ischemia region in the first brain image and the vessel occlusion, infarction or ischemia region in the second brain image. Specifically, the first brain image is the CT brain image, and the second brain image is the MRI brain image. Although the CT brain image has a low resolution with respect to the substantia nigra and the substantia alba, the CT brain image costs less time to be measured, being able to rapidly detect the vessel occlusion, infarction or ischemia region. On the other hand, the MRI brain image costs more time to be measured although the MRI brain image has a high resolution with respect to the substantia nigra and the substantia alba, which helps to find the long term vessel occlusion, infarction or ischemia region and pathological changes around the vessel occlusion, infarction or ischemia region. In short, the CT brain image and the MRI brain image help detect the vessel occlusion, infarction or ischemia region, but both have pros and cons. Therefore, the present disclosure uses the algorithms to generate the image of the vessel occlusion, infarction or ischemia region according to the vessel occlusion, infarction or ischemia region detected by the CT and the MRI. Additionally, the present disclosure uses a set-up application to automatically examine whether there is a vessel occlusion, infarction or ischemia in a patient's brain, so that the medical staff would not need to determine whether there is a vessel occlusion, infarction or ischemia in a patient's brain by observing the brain image.

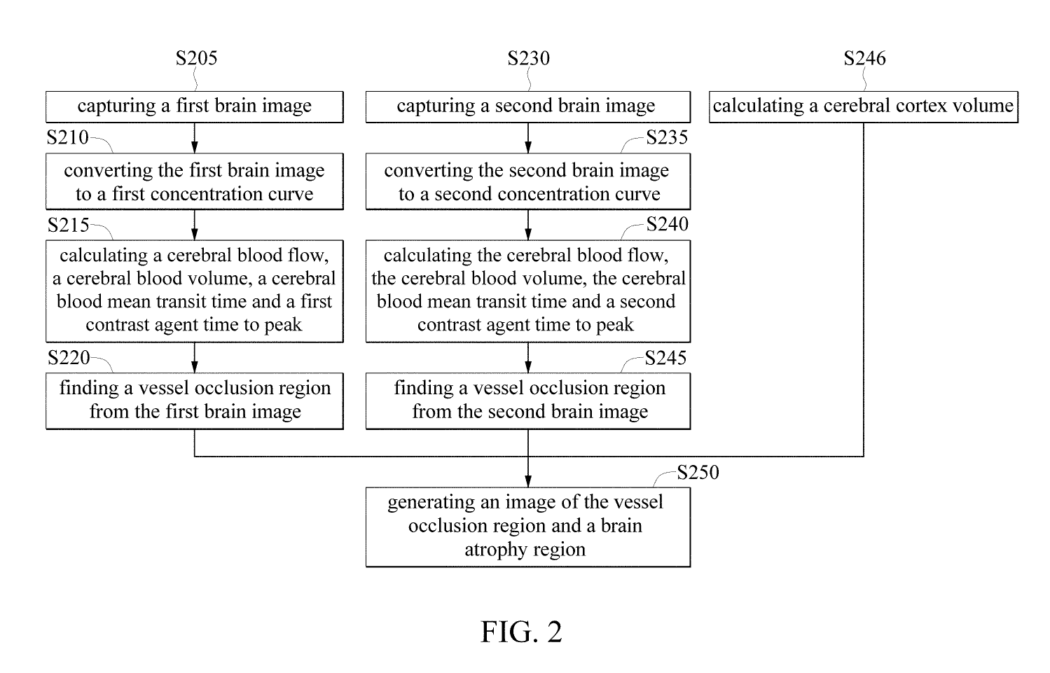

FIG. 2 shows a flow chart of a brain imaging method according to one embodiment of the present disclosure. The brain imaging method is adapted to the brain imaging system 100 to capture the brain image by using the contrast agent. According to FIG. 1A and FIG. 1B, the brain imaging system 100 detects the entrance 140 and the exit 150 of the brain where the contrast agent flows into and out from the brain. The brain imaging system 100 includes the first imaging device 110, the second imaging device 120, the third imaging device 135 and the central processor 130. The central processor 130 is coupled to the first imaging device 110, the second imaging device 120 and the third imaging device 135.

In step S205, the first imaging device 110 captures the first brain image. The first imaging device 110 is the CT imaging device, and the first brain image is the CT brain image.

In step S210, the first imaging device 110 converts the first brain image to the first concentration curve by the Hounsfield unit. The first concentration curve is the concentration curve of the iodinated contrast agent, and the first contrast agent time to peak is the iodinated contrast agent time to peak. In addition, the slope of the concentration curve of the iodinated contrast agents is positively proportional to the Hounsfield unit.

In step S215, the first imaging device 110 calculates the cerebral blood flow, the cerebral blood volume, the cerebral blood mean transit time and the first contrast agent time to peak according to the first concentration curve showing the concentrations at the positions of the entrance 140 and the exit 150. Also, the central processor 130 detects the position of the entrance 140 iodinated according to the iodinated contrast agent starting time, the iodinated contrast agent time to half-peak and the iodinated contrast agent time to peak.

In step S220, the central processor 130 detects the vessel occlusion, infarction or ischemia region of the first brain image according to one of the cerebral blood flow, the cerebral blood volume, the cerebral blood mean transit time and the first contrast agent time to peak. Specifically, when the cerebral blood flow is below 30% of the normal cerebral blood flow, the cerebral blood volume is smaller than 40% of the normal cerebral blood volume and the first contrast agent time to peak is increasing, the central processor 130 through the first imaging device 110 detects the infarct core of the vessel occlusion, infarction or ischemia region in the first brain image. In addition, when the cerebral blood flow is decreasing, the cerebral blood volume is maintained or increased, and the first contrast agent time to peak is dramatically increasing, the central processor 130 through the first imaging device 110 detects the penumbra of the vessel occlusion, infarction or ischemia region in the first brain image.

In step S230, the second imaging device 120 captures the second brain image. The second imaging device 120 is the MRI device, and the second brain image is the MRI brain image.

In step S235, the second imaging device 120 converts the second brain image to the second concentration curve through an equation (shown in the above embodiment). The second concentration curve is the concentration curve of the Gadolinium contrast agent, and the second contrast agent time to peak is the Gadolinium contrast agent time to peak.

In step S240, the second imaging device 120 calculates the cerebral blood flow, the cerebral blood volume, the cerebral blood mean transit time and the second contrast agent time to peak according to the second concentration curve showing the concentrations at the positions of the entrance 140 and the exit 150. Also, the central processor 130 detects the position of the entrance 140 where the Gadolinium contrast agent flows into the brain according to the Gadolinium contrast agent starting time, an Gadolinium contrast agents time to half-peak and the Gadolinium contrast agent time to peak.

In step S245, the central processor 130 detects the vessel occlusion, infarction or ischemia region of the second brain image according to one of the second concentration curve, the cerebral blood flow, the cerebral blood volume, the cerebral blood mean transit time and the second contrast agent time to peak.

In step S246, the third imaging device 135 uses the FSL software to capture the third brain image. The third brain image is a structural brain image, and the structural brain image is the T₁ image. In addition, the third imaging device 135 can be also configured to calculate the cerebral cortex volume to obtain the brain atrophy region.

In step S250, the central processor 130 uses the algorithms to generate the images of regions with the vessel occlusion, infarction or ischemia according to the vessel occlusion, infarction or ischemia region in the first brain image and the vessel occlusion, infarction or ischemia region in the second brain image. In addition, the third imaging device 150 calculates the brain atrophy region according to the cerebral cortex volume.

To sum up, in the present disclosure, the CT brain image and the MRI brain image are generated respectively by the first imaging device and the second imaging device. Then, the CT brain image and the MRI brain image are converted into the concentration curves to calculate the cerebral blood flow, the cerebral blood volume, the cerebral blood mean transit time and the contrast agent time to peak. After that, the central processor detects the vessel occlusion, infarction or ischemia region in the CT brain image, and the vessel occlusion, infarction or ischemia region and regions where blood flows are affected in the MRI brain image according to the cerebral blood flow, the cerebral blood volume, the cerebral blood mean transit time and the contrast agent time to peak. In addition, the third imaging device calculates the cerebral cortex volume to determine whether a specific brain region has obvious atrophy or affections. The images of regions with the vessel occlusion, infarction or ischemia and the brain atrophy region are generated by algorithms to improve the conventional way of determining the positions of the vessel occlusion, infarction or ischemia region and the brain atrophy region. Therefore, the present disclosure effectively improves the efficiency and the precision of the examination of the brain vessel occlusion and dementia.

The descriptions illustrated supra set forth simply the preferred embodiments of the present disclosure; however, the characteristics of the present disclosure are by no means restricted thereto. All changes, alterations, or modifications conveniently considered by those skilled in the art are deemed to be encompassed within the scope of the present disclosure delineated by the following claims.

What is claimed is:

1. A brain imaging system, capturing a brain image by using contrast agents, wherein the brain imaging system detects positions of an entrance and an exit where the contrast agents flow into and out from a brain, and comprises:
a first imaging device, configured to capture a first brain image, convert the first brain image to a first concentration curve by Hounsfield Unit, and calculate a cerebral blood flow, a cerebral blood volume, a cerebral blood mean transit time and a first contrast agent time to peak according to the first concentration curve showing concentrations at the positions of the entrance and the exit;
a second imaging device, configured to capture a second brain image, convert the second brain image to a second concentration curve through an equation, and calculate the cerebral blood flow, the cerebral blood volume, the cerebral blood mean transit time and a second contrast agent time to peak according to the second concentration curve showing concentrations at the positions of the entrance and the exit;
a third imaging device, configured to calculate a cerebral cortex volume; and
a central processor, coupled to the first imaging device, the second imaging device and the third imaging device, configured to detect a vessel occlusion, infarction or ischemia region of the first brain image according to at least one of the cerebral blood flow, the cerebral blood volume, the cerebral blood mean transit time and the first contrast agent time to peak, and detect a vessel occlusion, infarction or ischemia region of the second brain image according to at least one of the second concentration curve, the cerebral blood flow, the cerebral blood volume, the cerebral blood mean transit time and the second contrast agent time to peak;
wherein the central processor generates an image of the vessel occlusion, infarction or ischemia region respectively according to the vessel occlusion, infarction or ischemia region in the first brain image and the vessel occlusion, infarction or ischemia region in the second brain image, and the third imaging device obtains a brain atrophy region according to the cerebral cortex volume.

2. The brain imaging system according to claim 1, wherein the first imaging device is a computed tomography (CT) imaging device, the first brain image is a CT brain image, the first concentration curve is a concentration curve of an iodinated contrast agent, and the first contrast agent time to peak is an iodinated contrast agent time to peak;
wherein a slope of the concentration curve of the iodinated contrast agent is positively proportional to the Hounsfield unit, and the central processor detects the position of the entrance where the iodinated contrast agent flows into the brain according to an iodinated contrast agent starting time, an iodinated contrast agent time to half-peak and the iodinated contrast agent time to peak.

3. The brain imaging system according to claim 1,
wherein when the cerebral blood flow is below 30% of a normal cerebral blood flow, the cerebral blood volume is smaller than 40% of a normal cerebral blood volume and the first contrast agent time to peak is increasing, the central processor detects an infarct core of the vessel occlusion, infarction or ischemia region in the first brain image via the first imaging device;
wherein when the cerebral blood flow is decreasing, the cerebral blood volume is maintained or increased, and the first contrast agent time to peak is dramatically increasing, the central processor detects a penumbra of the vessel occlusion, infarction or ischemia region in the first brain image via the first imaging device.

4. The brain imaging system according to claim 1, wherein the second imaging device is a magnetic resonance imaging (MRI) device, the second brain image is an MRI brain image, the second concentration curve is a concentration curve of a Gadolinium contrast agent, the second contrast agent time to peak is a Gadolinium contrast agent time to peak, and the central processor generates the position of the entrance where the Gadolinium contrast agent flows into the brain according to an Gadolinium contrast agent starting time, an Gadolinium contrast agent time to half-peak and the Gadolinium contrast agent time to peak.

5. The brain imaging system according to claim 1, wherein the equation is
$$S ( x , y , b ) = {M}_{0} ( 1 - {e}^{- \frac{TR}{{T}_{1} ( x , y )}} ) {e}^{- \frac{TE}{{T}_{2}^{*} ( x , y )}} {e}^{- ADC \cdot b}$$
wherein T₁ is a spin-lattice relaxation time, T₂* is a transverse relaxation time, TR is a cycle time, TE is an echo time, b is a setting parameter of an imaging device, (x, y) is the position of the brain image, Mo is an initial value of the brain image, and the equation includes an apparent diffusion coefficient (ADC) and the ADC is
$$ADC ( x , y ) = - \frac{1}{b} ln ( \frac{S ( x , y , 1000 )}{S ( x , y , 0 )} ) {mm}^{2} \text{/} s$$
wherein when the ADC is smaller than a diffusion threshold, the central processor detects an infarct core of the vessel occlusion, infarction or ischemia region in the second brain image by using the second imaging device, and when the second contrast agent time to peak is larger than a time to peak, the central processor detects a penumbra of the vessel occlusion, infarction or ischemia region in the second brain image by using the second imaging device;
wherein the central processor calculates the diffusion threshold and the time to peak based on Bayesian Statistics;
wherein the central processor captures a calvarium image of the second brain image by using a FMRIB Software Library (FSL) software to execute a Brain Extraction Tool, the central processor separates the calvarium image from the second brain image and divides the second brain image without the calvarium image into a plurality of brain regions, the central processor detects the position of the vessel occlusion, infarction or ischemia region in the second brain image based on the Bayesian Statistics, and a third brain image captured by the central processor by using the FSL software is a structural brain image.

6. A brain imaging method, adapted to a brain imaging system for capturing a brain image by using contrast agents, wherein the brain imaging system detects positions of an entrance and an exit where the contrast agents flow into and out from a brain, the brain imaging system includes a first imaging device, a second imaging device, a third imaging device and a central processor, the central processor is coupled to the first imaging device, the second imaging device and the third imaging device, and the brain imaging method comprises:
through the first imaging device, capturing a first brain image;
through the first imaging device, converting the first brain image to a first concentration curve by Hounsfield Unit;
through the first imaging device, calculating a cerebral blood flow, a cerebral blood volume, a cerebral blood mean transit time and a first contrast agent time to peak according to the first concentration curve showing concentrations at the positions of the entrance and the exit;
through the second imaging device, capturing a second brain image;
through the second imaging device, converting the second brain image to a second concentration curve through an equation;
through the second imaging device, calculating the cerebral blood flow, the cerebral blood volume, the cerebral blood mean transit time and a second contrast agent time to peak according to the second concentration curve showing concentrations at the positions of the entrance and the exit;
through the third imaging device, calculating a cerebral cortex volume;
through the central processor, detecting a vessel occlusion, infarction or ischemia region of the first brain image according to at least one of the cerebral blood flow, the cerebral blood volume, the cerebral blood mean transit time or the first contrast agent time to peak; and
through the central processor, detecting a vessel occlusion, infarction or ischemia region of the second brain image according to at least one of the second concentration curve, the cerebral blood flow, the cerebral blood volume, the cerebral blood mean transit time and the second contrast agent time to peak;
wherein the central processor generates an image of the vessel occlusion, infarction or ischemia region respectively according to the vessel occlusion, infarction or ischemia region in the first brain image and the vessel occlusion, infarction or ischemia region in the second brain image, and the third imaging device obtains a brain atrophy region according to the cerebral cortex volume.

7. The brain imaging method according to claim 6, wherein the first imaging device is a computed tomography (CT) imaging device, the first brain image is a CT brain image, the first concentration curve is a concentration curve of an iodinated contrast agent, the first contrast agent time to peak is an iodinated contrast agent time to peak, a slope of the concentration curve of the iodinated contrast agent is positively proportional to the Hounsfield unit, and the brain imaging method further comprises:
through the central processor, detecting the position of the entrance where the iodinated contrast agent flows into the brain according to an iodinated contrast agent starting time, an iodinated contrast agent time to half-peak and the iodinated contrast agent time to peak.

8. The brain imaging method according to claim 6, further comprising:
wherein when the cerebral blood flow is below 30% of a normal cerebral blood flow, the cerebral blood volume is smaller than 40% of a normal cerebral blood volume and the first contrast agent time to peak is increasing, the central processor detects an infarct core of the vessel occlusion, infarction or ischemia region in the first brain image via the first imaging device;
wherein when the cerebral blood flow is decreasing, the cerebral blood volume is maintained or increased, and the first contrast agent time to peak is dramatically increasing, the central processor detects a penumbra of the vessel occlusion, infarction or ischemia region in the first brain image.

9. The brain imaging method according to claim 6, wherein the second imaging device is a magnetic resonance imaging (MRI) device, the second brain image is an MRI brain image, the second concentration curve is a concentration curve of a Gadolinium contrast agent, the second contrast agent time to peak is a Gadolinium contrast agent time to peak, and the brain imaging method further comprises:
through the central processor, generating the position of the entrance where the Gadolinium contrast agent flows into the brain according to an Gadolinium contrast agent starting time, an Gadolinium contrast agent time to half-peak and the Gadolinium contrast agent time to peak.

10. The brain imaging method according to claim 6, wherein the equation is
$$S ( x , y , b ) = {M}_{0} ( 1 - {e}^{- \frac{TR}{{T}_{1} ( x , y )}} ) {e}^{- \frac{TE}{{T}_{2}^{*} ( x , y )}} {e}^{- ADC \cdot b} ,$$
T₁ is a spin-lattice relaxation time, T₂* is a transverse relaxation time, TR is a cycle time, TE is an echo time, b is a setting parameter of an imaging device, (x, y) is the position of the brain image, Mo is an initial value of the brain image, the equation includes an apparent diffusion coefficient (ADC) and the ADC is
$$ADC ( x , y ) = - \frac{1}{b} ln ( \frac{S ( x , y , 1000 )}{S ( x , y , 0 )} ) {mm}^{2} \text{/} s ,$$
and the brain imaging method further comprises:
wherein when the ADC is smaller than a diffusion threshold, the central processor detects an infarct core of the vessel occlusion, infarction or ischemia region in the second brain image by using the second imaging device, and when the second contrast agent time to peak is larger than a time to peak, the central processor detects a penumbra of the vessel occlusion, infarction or ischemia region in the second brain image by using the second imaging device;
wherein the central processor calculates the diffusion threshold and the time to peak based on Bayesian statistics;
wherein the central processor captures a calvarium image of the second brain image by using a FMRIB Software Library (FSL) software to execute a Brain Extraction Tool, the central processor separates the calvarium image from the second brain image and divides the second brain image without the calvarium image into a plurality of brain regions, the central processor detects the position of the vessel occlusion, infarction or ischemia region in the second brain image based on the Bayesian statistics, and a third brain image captured by the central processor by using the FSL software is a structural brain image.
